(19) United States
(12) Patent Application Publication
(10) Pub. No.: US 2013/0096555 A1
(43) Pub. Date: Apr. 18, 2013

(54) ELECTRICAL CHARGE-DISSIPATING CANNULA

(71) Applicant: INTUITIVE SURGICAL OPERATIONS, INC., Sunnyvale, CA (US)

(72) Inventors: Justin KROM, Southington, CT (US); Joseph P. ORBAN, III, Norwalk, CT (US); Salvatore J. BROGNA, Pleasanton, CA (US); Augusto V. CASTELLO, San Jose, CA (US)

(73) Assignee: INTUITIVE SURGICAL OPERATIONS, INC., Sunnyvale, CA (US)

(21) Appl. No.: 13/650,770

(22) Filed: Oct. 12, 2012

Related U.S. Application Data
(60) Provisional application No. 61/546,781, filed on Oct. 13, 2011.

(51) Int. Cl. A61B 18/14

(52) U.S. Cl. 606/41

(57) ABSTRACT

An apparatus, a system and a method of dissipating an electrical charge are provided. The apparatus is a cannula for receiving a surgical instrument to perform a surgical operation on a body. The cannula includes a hollow elongated structure having a proximal end opening and a distal end opening leading to a hollow interior passage dimensioned to receive a surgical instrument. The hollow elongated structure includes a polymer material and an electrically conductive material. The electrically conductive material is disposed to achieve electrical capacitive coupling with the surgical instrument and to dissipate an electrical charge received via the electrical capacitive coupling. The system and method utilize the cannula to dissipate the electrical charge through the surgical instrument.

### CROSS-REFERENCE TO RELATED APPLICATIONS

This application claims the benefits of priority of U.S. Provisional Patent Application No. 61/546,781, entitled “Electrical Charge-Dissipating Cannula,” filed on Oct. 13, 2011, which is incorporated by reference herein in its entirety.

### TECHNICAL FIELD

The present disclosure is generally directed to surgical instrument equipment. More particularly, aspects of the present disclosure relate to an apparatus to dissipate electrical charge from surgical instruments.

### INTRODUCTION

Surgical instruments are used in both manual and robotic surgery (an example of the latter including da Vinci® robotic surgical systems, commercialized by Intuitive Surgical, Inc., Sunnyvale, Calif.) and typically utilize additional devices for support and/or stabilization, such as cannulas, stabilizers, trocars, ports and the like, when performing surgical operations. These additional surgical devices may receive a surgical instrument, either manual or robotic, therein to allow a surgeon to access a patient surgical site.

Cannulas, which are supporting devices that are configured to receive the surgical instruments, are provided either directly at the surgical site on the patient or in close proximity to the surgical site, e.g., above the operation site of the patient. Various surgical instruments may receive from an energy source an electric current that is delivered through the surgical instrument to a live electrical element at a distal end of the instrument. The electrical element can then deliver the electrical energy at a desired surgical site to perform, for example, procedures such as cauterization, ablation, etc. In robotic surgical instruments, for example, the electrical charge provided through the instrument may be up to about 4500 volts, for example. In some instances, the electric current from the instrument can capacitively couple to other electrically conductive material, such as, for example, portions of the surgical instrument other than the live electrical element, the cannula and/or robotic arms used to control the movement of the surgical instrument. If this built-up charge suddenly discharges from the conductive material, the charge may flow to a patient's tissue at an undesirable location. This can cause burning of the patient at a site other than the intended surgical site (“alternate site burning”) and/or burning of an assistant in contact with the component.

In manual laparoscopic procedures, surgical instruments, such as monopolar electrosurgical instruments, are commonly used through cannulas, trocars and/or ports made from a substantially electrically insulative material, such as plastic for example. The surgical instruments are provided with electrical insulation over all components except the live electrical element, e.g., the conductor wire or push rod, at the distal end that operates to perform the surgical procedure relying on the delivery of electrical energy. Thus, the patient is kept safe as long as the insulation stays intact.

Various robotic laparoscopic procedures employ metal wristed or jointed surgical instruments. For at least some procedures, it is desirable to make the wrists relatively small, for example having relatively small lateral dimensions (e.g., diameters) on the order of several millimeters (e.g., ranging from about 5 mm to about 8 mm or less) for a variety of procedures, such as, for example, general surgery procedures, gynecological procedures, such as a hysterectomy, and pediatric procedures, to name a few examples. In turn, it also is desirable to deliver such surgical instruments through cannulas and/or other support devices that also have relatively small diameters such as, for example, cannulas having inner diameters ranging from about 5 mm to about 8 mm, and in some cases about 5 mm or less. In such circumstances, the inner diameter of the cannula may not be large enough to accommodate a surgical instrument covered with electrical insulation, such as a sleeve over the electrically conductive wrist or elsewhere along the surgical instrument, to protect against the unwanted discharge of electrical charge from the surgical instrument. Therefore, cannulas commonly used in such procedures are metal to dissipate the electrical charge that may build up on the surgical instrument, and the metal cannula is directly attached to the body wall of the patient. The patient is set on a dispersive electrode, which is connected back to the generator that supplies the electrical energy to the surgical instrument, thereby completing the circuit. Any electrical charge that builds up on the surgical instrument due to the use of the live electrical element is then transferred to the cannula and dissipated through the body wall back to the generator.

Thus, for surgical instruments, in particular, robotic surgical instruments that may include exposed metal parts other than the live electrical element, such as wrists or joints, when the cannula being used is metal, an electrical charge that forms on the exposed metal parts does not arc or otherwise flow to undesired locations on the patient. Rather, the energy is capacitively coupled to the metal cannula and is bled off to the body wall of the patient.

In some applications, it may be desirable to use a cannula or other support device made from plastic, in order, for example, to reduce the cost of fabrication and/or sterilization, which thereby may allow for disposability of a single-use cannula or other support device. However, conventional cannulas made of plastic are not configured to achieve capacitive coupling and dissipation of an electrical charge that builds up on the exposed electrically conductive parts of the surgical instrument (e.g., a wrist). Accordingly, the risk of alternate site burning exists unless the exposed metal parts, such as wrists or joints, are protected by electrically insulative covers or the like. As discussed above, however, for applications in which minimizing overall dimensions of the surgical instruments and supporting devices is desired, providing such coverings may not be feasible. For example, providing effective electrical insulation may increase the size of the instrument to an unacceptable degree. Alternatively, if a thinner electrical insulation is used, it may wear down or fail.

Additionally, in surgical operations, sterilization of the equipment is of utmost concern. Disposable materials intended for single use are therefore desirable to minimize the costs associated with sterilizing equipment between uses. Metal cannulas, which are not typically disposable in light of the relative expense of fabrication, are thus generally used on multiple patients. While metal cannulas provide the ability to dissipate an electrical charge, particularly in situations in which the surgical instrument is not insulated or the insulation tends to break down, e.g., at a wrist or a joint, the metal cannulas require additional handling in order to effectively clean and sterilize the cannulas between patient use. In turn, additional costs associated with the sterilization process are incurred when using metal cannulas. In addition, in some cases, it may be desirable to provide cannulas with insufflation ports or other passages, which structures pose challenges to effectively sterilize.

Moreover, metal cannulas also prohibit surgeons from the ability to see through the cannulas to determine, for example, the amount of insertion of the surgical instrument.

There exists a need, therefore, to provide cannulas and other surgical instrument supporting devices to address one or more of the various drawbacks noted above and provide features that are not presently met by existing cannulas.

### SUMMARY

The present disclosure may solve one or more of the problems and/or may demonstrate one or more of the desirable features set forth herein. Other features and/or advantages may become apparent from the description that follows.

In accordance with at least one exemplary embodiment, a cannula for receiving a surgical instrument to perform a surgical operation on a body comprises a hollow elongated structure having a proximal end opening and a distal end opening leading to a hollow interior passage dimensioned to receive a surgical instrument. The hollow elongated structure comprises a polymer material and an electrically conductive material. The electrically conductive material is disposed to achieve electrical capacitive coupling with the surgical instrument and to dissipate an electrical charge received via the electrical capacitive coupling.

In accordance with at least one exemplary embodiment, the present teachings contemplate a system to dissipate an electrical charge from a surgical instrument. The system includes a cannula, comprising a hollow elongated structure to receive a surgical instrument and an electrically conductive material. At least a portion of the hollow elongated structure comprises a polymer material. The electrically conductive material is provided at the hollow elongated structure sufficient to receive an electrical charge from the surgical instrument through capacitive coupling between the surgical instrument and the hollow elongated structure. The system also includes at least one dispersive electrode configured to be placed in contact with a patient's body.

In accordance with at least one exemplary embodiment, the present teachings contemplate a method of dissipating an electrical charge from a surgical instrument. The method includes connecting a cannula to a body in contact with at least one dispersive electrode. The cannula has an electrically conductive material that is sufficient to receive an electrical charge from the surgical instrument through capacitive coupling between the surgical instrument and the cannula. At least a portion of the cannula comprises a polymer material. The method further includes inserting the surgical instrument within an interior passage of the cannula, and providing energy to the surgical instrument from an energy source. The electrical charge is dissipated through the electrically conductive material of the cannula to the body after the surgical instrument is provided with energy from the energy source.

Additional objects and advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are exemplary and explanatory only and are not restrictive of the invention, as claimed; rather the claims should be entitled to their full breadth of scope, including equivalents.

### BRIEF DESCRIPTION OF THE DRAWINGS

The present disclosure can be understood from the following detailed description either alone or together with the accompanying drawings. The drawings are included to provide a further understanding of the present disclosure, and are incorporated in and constitute a part of this specification. The drawings, which are incorporated in and constitute a part of this specification, illustrate one or more embodiments of the present disclosure and, together with the description, serve to explain certain principles and operation. In the drawings,

FIG. 1A is a proximal end view of a cannula in accordance with at least one exemplary embodiment of the present disclosure;

FIG. 1B is a side, cross-sectional view of the cannula taken along lines 1B-1B, shown in FIG. 1A;

FIG. 2A is a proximal end view of a cannula in accordance with at least one exemplary embodiment of the present disclosure;

FIG. 2B is a side, cross-sectional view of the cannula taken along lines 2B-2B, shown in FIG. 2A, in accordance with at least one exemplary embodiment of the present disclosure;

FIG. 2C is a partial, side detailed view of the cannula taken along lines 2B-2B, shown in FIG. 2A, in accordance with at least one exemplary embodiment of the present disclosure;

FIG. 3A is a proximal end view of a cannula in accordance with at least one exemplary embodiment of the present disclosure;

FIG. 3B is a side, cross-sectional view of the cannula taken along lines 3B-3B, shown in FIG. 3A;

FIG. 4A is a proximal end view of a cannula in accordance with at least one exemplary embodiment of the present disclosure;

FIG. 4B is a side, cross-sectional view of the cannula taken along lines 4B-4B, shown in FIG. 4A;

FIG. 5 is a diagrammatic view of a system for dissipating an electrical charge in accordance with at least one exemplary embodiment of the present disclosure;

FIG. 6 a diagrammatic view of a system for dissipating an electrical charge in accordance with at least one exemplary embodiment of the present disclosure; and

FIG. 7 is a diagrammatic view of a patient side console of an exemplary robotic surgical system in accordance with at least one exemplary embodiment of the present disclosure.

### DETAILED DESCRIPTION

The following description and the accompanying drawings illustrate exemplary embodiments and should not be taken as limiting, with the claims defining the scope of the present disclosure, including equivalents. Various mechanical, compositional, structural, electrical, and operational changes may be made without departing from the scope of this description and the invention as claimed, including equivalents. In some instances, well-known structures, and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or the electrosurgical instrument.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Various exemplary embodiments provide an inexpensive, and therefore disposable, surgical supporting device, such as, for example, a cannula, wherein at least a portion of the cannula is made from a polymer material and at least a portion is made from electrically conductive material that effectively dissipates an electric charge from a surgical instrument inserted into the cannula, in particular, in surgical instruments that include metal parts for which insulation may not be provided or may not totally eliminate capacitive coupling charges. Various exemplary embodiments thus provide a cannula or other surgical instrument supporting device made of materials having a lower cost, particularly in comparison to cannulas made entirely of metal. Various exemplary embodiments also provide a cannula which may be transparent, to allow a surgeon, for example, to see through the cannula. Features of the exemplary embodiments additionally provide a cannula or other surgical instrument supporting device that is suitable for relatively small diameter applications, such as, for example, for use with surgical instruments of 5-8 mm or less, while also being able to effectively dissipate an electrical charge. In addition, various exemplary embodiments provide a system and method of dissipating an electric charge from a surgical instrument.

Further, some cannulas include a bowl having a seal to prevent insufflation from leaking out of the cannula when no instrument is received within the cannula, and when there is an instrument received within the cannula, the seal at the bowl seals the instrument to the cannula. Due to the nature of the seal, the bowl is more expensive to make than, for example, a tube section, connected to the bowl, through which the surgical instrument extends. Thus, various exemplary embodiments provide a cannula where the bowl is made of a relatively inexpensive material in order to minimize the costs associated with providing a disposable cannula. In addition, various exemplary embodiments provide a cannula with a bowl made from a material, such as a polymer, that lends itself to integrating a seal relatively easily.

Although the exemplary embodiments and description below relate to cannulas, the principles of the exemplary embodiments could be applied to other supporting devices and/or surgical tools, including but not limited to, for example, stabilizers, trocars, ports, and the like for surgical instruments, or for non-surgical devices and applications that may benefit from capacitive coupling and dissipating electrical charge.

In addition, the cannulas according to the exemplary embodiments may be used with either robotic surgical instruments or non-robotic surgical instruments, such as manual laparoscopic surgical instruments.

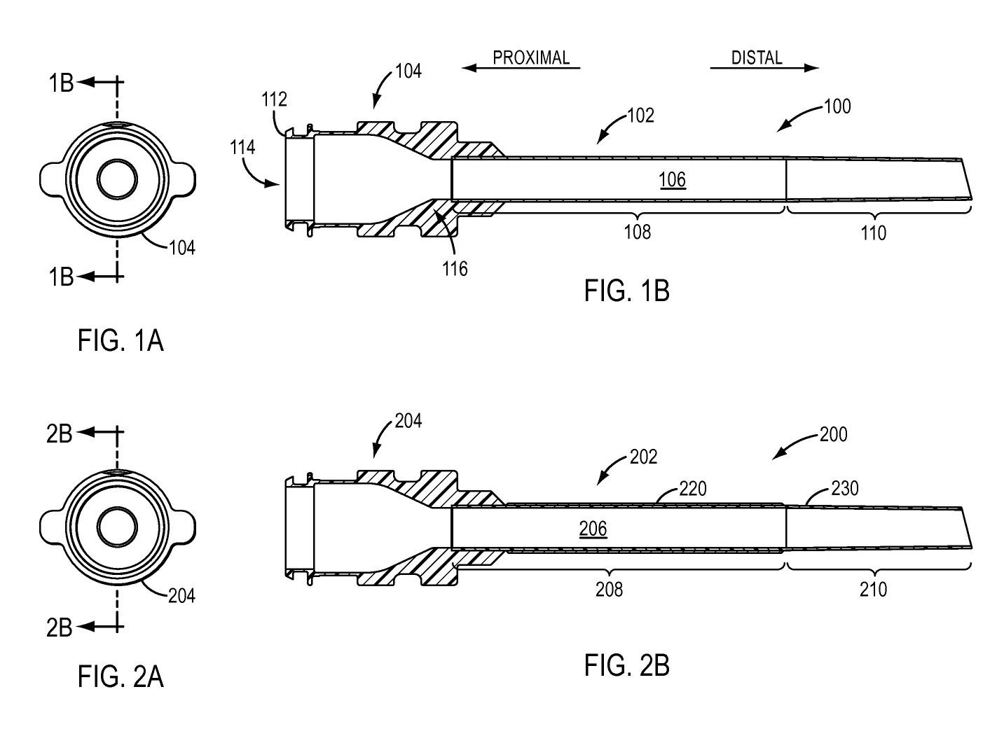

With reference to FIGS. 1A-1B, 2A-2B, 3A-3B, and 4A-4B, which are proximal end views, and side elevation cross-sectional views, respectively, cannulas in accordance with exemplary embodiments of the present disclosure are depicted. The directions “proximal” and “distal” are used herein to define the directions as illustrated in FIG. 1B, with distal generally being in a direction further along the cannula or closest to the surgical work site in the intended operational use, for example, in use for performing surgical procedures. FIGS. 1A and 1B show the cannula 100 in accordance with at least one embodiment. The cannula 100 has a hollow elongated structure that includes a tube section 102 and a bowl 104 connected at a proximal end of the tube section 102.

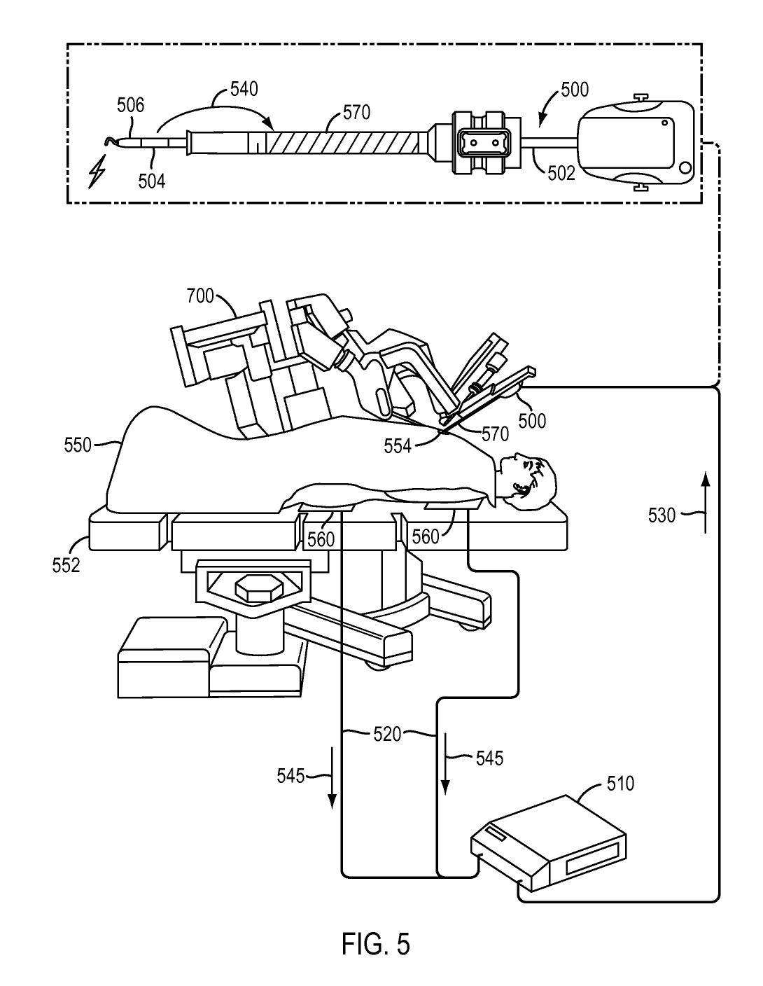

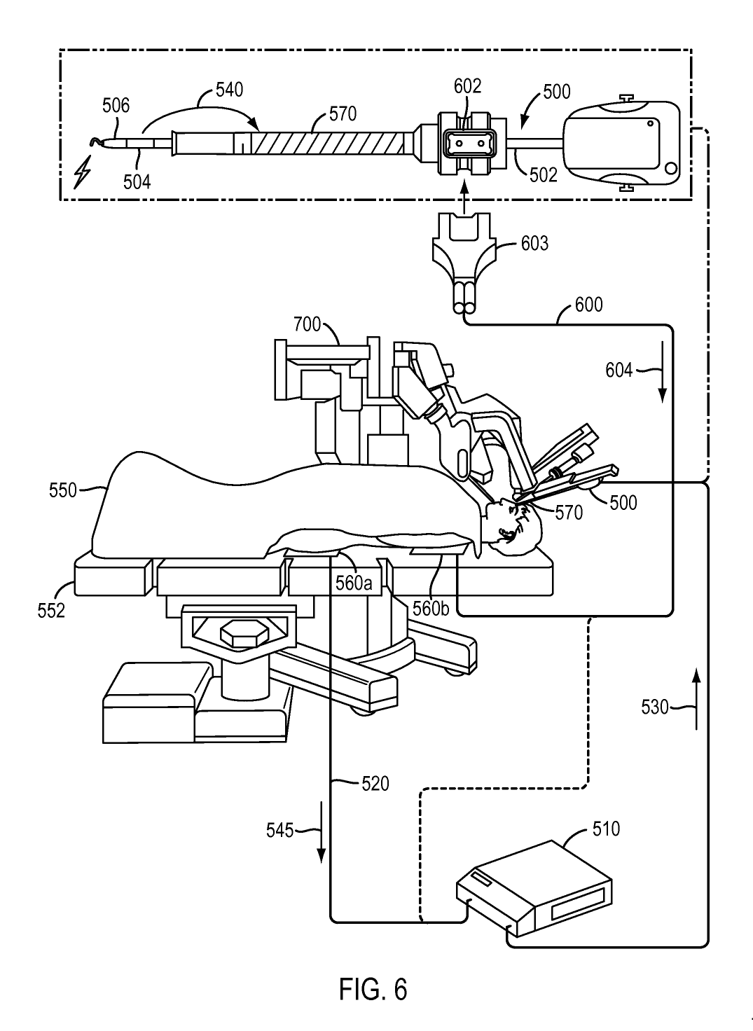

The tube section 102 has an interior passage 106 defined therein, through which a surgical instrument 500, shown in FIGS. 5 and 6, for example, can be received. In the exemplary embodiment shown, the tube section 102 includes a proximal main portion 108 and a tapered distal portion 110 integrally connected with the proximal main portion 108. In accordance with at least one exemplary embodiment, the tube section 102 is made from an electrically conductive material. The electrically conductive material may be, for example, stainless steel or other metal. The conductive material may be provided at the proximal main portion 108, at the tapered distal portion 110, or at both the proximal main portion 108 and the tapered portion 110. The electrically conductive material is disposed in a location and amount sufficient to allow electrical capacitive coupling of the cannula with a surgical instrument and the dissipation of the electrical capacitive charge, for example, so as to avoid alternate site burning, as will be discussed below in more detail with reference to FIGS. 5 and 6.

The bowl 104 includes a proximal end 112 with an opening 114 through which the surgical instrument 500 can be introduced into the cannula 100. In accordance with at least one exemplary embodiment, the bowl 104 is made from a polymer material that is substantially electrically insulative. It is to be understood that the term “substantially electrically insulative” refers to the material of the bowl providing a high enough level of electrical insulation such that the material would not receive an electrical charge nor dissipate an electrical charge. The polymer material may include but is not limited to, for example, a plastic such as, for example, acrylic, polycarbonate, polyetherimide, polyether ether ketone, and/or other similar materials and combinations thereof. The bowl 104 includes a distal end 116 adjacent the interior passage 106 of the tube section 102.

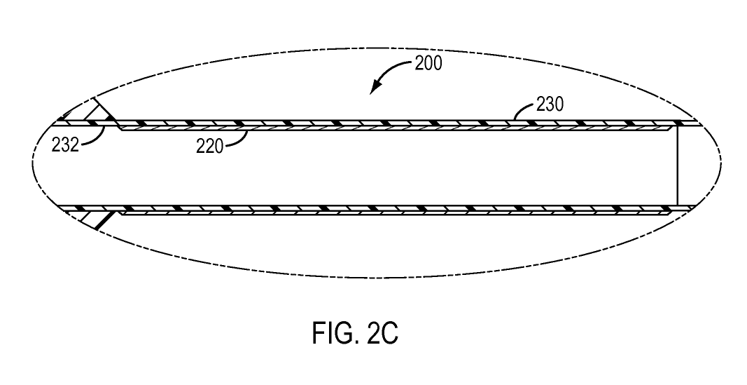

FIGS. 2A and 2B show a cannula 200 in accordance with at least one embodiment of the present disclosure. The cannula 200 has a hollow elongated structure that includes a tube section 202 and a bowl 204 connected at a proximal end of the tube section 202. The tube section 202 has an interior passage 206 defined therein, through which a surgical instrument 500, shown in FIGS. 5 and 6, for example, can be received. In the exemplary embodiment shown, the tube section 202 includes a proximal main portion 208 and a tapered distal portion 210. In accordance with at least one embodiment, the tube section 202 is made from a polymer material, such as, for example, plastic. In various exemplary embodiments, the tube section 202 may include a substantially electrically insulative material, for example, chosen from acrylic, polycarbonate, polyetherimide, poyether ether ketone, and/or similar materials and combinations thereof. In addition, the tube section 202 includes one or more electrically conductive layers 220 provided on at least one of an exterior surface 230 (FIG. 2B) or an interior surface 232 (FIG. 2C) of the polymer material. Although FIG. 2B depicts two layers 220, it should be understood that the layer 220 may be on either the interior surface 232, as in FIG. 2C, the exterior surface 230, as in FIG. 2B, or on both the interior surface 232 and the exterior surface 230 of the polymer material of the tube section 202.

In various exemplary embodiments, the one or more layers 220 may be formed on the polymer material of the tube section 202 via plating (e.g., electrolytic or electroless plating), coating or the like. In various exemplary embodiments, the electrically conductive layers 220 may be copper, chrome, gold, or other similar electrically conductive material suitable for application to a polymer (e.g., plastic) via plating or coating. The one or more electrically conductive layers 220 may be provided at the proximal main portion 208, at the tapered portion 210, or at both the proximal main portion 208 and the tapered portion 210. In various exemplary embodiments, a layer 220 may have a thickness ranging from about 0.0001 inch to about 0.010 inch. The layer 220 may extend substantially the entire length of the tube section 202 or for a portion of the tube section 202. For example, in various exemplary embodiments, the layer 220 may extend a length ranging from about 0.500 inch to about 5.00 inches. The one or more electrically conductive layers 220 are disposed in a location and amount sufficient to allow electrical capacitive coupling of the cannula with a surgical instrument and the dissipation of the electrical capacitive charge, for example, so as to avoid arcing and alternate site burning, as will be discussed below in more detail with reference to FIGS. 5 and 6.

In accordance with at least one exemplary embodiment, the bowl 204 is made of a polymer material. The bowl section 204 need not be configured to be electrically conductive, and can be substantially electrically insulative in various exemplary embodiments.

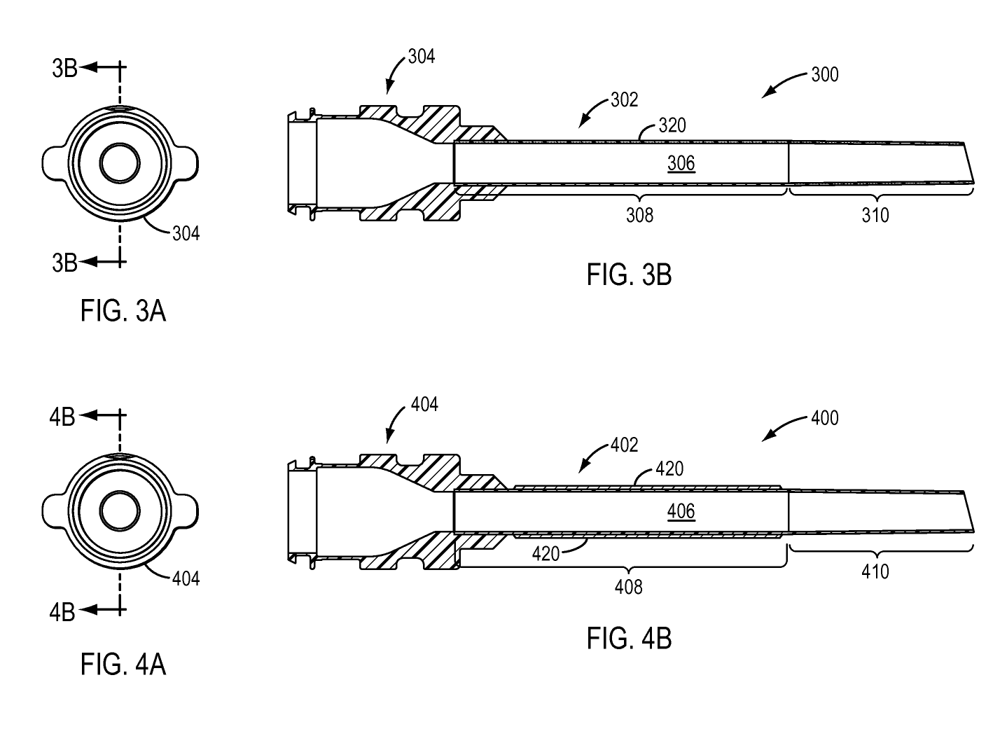

FIGS. 3A and 3B show a cannula 300 in accordance with at least one embodiment of the present disclosure. The cannula 300 has a hollow elongated structure that includes a tube section 302 and a bowl 304 connected at a proximal end of the tube section 302. The tube section 302 has an interior passage 306 defined therein, through which a surgical instrument 500, shown in FIGS. 5 and 6, for example, can be received. In the exemplary embodiment shown, the tube section 302 includes a proximal main portion 308 and a tapered distal portion 310. In accordance with at least one embodiment, the tube section 302 is made from an electrically conductive composite material 320. The conductive composite material 320 is composed of a polymer material, e.g., plastic, matrix with an electrically conductive material dispersed therein. In various exemplary embodiments, the electrically conductive material can be chosen from carbon or graphite, but is not limited thereto and may be any type of electrically conductive material able to be dispersed within the polymer material. For example, suitable materials for the tube section include, but are not limited to, electrically conductive composites of acrylonitrile butadiene styrene (ABS), polycarbonate, nylon, liquid crystal polymer, epoxy, and graphite. The conductive material may be in particulate form, including but not limited to, for example, fibers, powder or other similar form. The composite polymer material matrix having the electrically conductive material dispersed therein can provide an electrically conductive composite material 320 that is stiffer and/or stronger than the polymer material alone. The conductive composite material 320 may be provided at the proximal main portion 308, at the tapered portion 310, or at both the proximal main portion 308 and the tapered distal portion 310. The conductive composite material 320 is disposed in a location and amount sufficient to allow electrical capacitive coupling of the cannula with a surgical instrument and the dissipation of the electrical capacitive charge, for example, so as to avoid alternate site burning, as will be discussed below in more detail with reference to FIGS. 5 and 6. In accordance with at least one exemplary embodiment, the bowl 304 is made from a polymer material.

FIGS. 4A and 4B show a cannula 400 in accordance with yet another embodiment of the present disclosure. The cannula 400 has a hollow elongated structure that includes a tube section 402 and a bowl 404 connected at a proximal end of the tube section 402. The tube section 402 has an interior passage 406 defined therein, through which a surgical instrument 500, shown in FIGS. 5 and 6, for example, can be received. In the exemplary embodiment shown, the tube section 402 includes a proximal main portion 408 and a tapered distal portion 410. In accordance with at least one embodiment, the tube section 402 is made from a polymer material, such as, for example, a plastic. In various exemplary embodiments, the tube section 402 material may be substantially electrically insulative, and for example, may be chosen from acrylic, polycarbonate, polyetherimide, poyether ether ketone, and/or similar materials and combinations thereof. An electrically conductive sleeve 420 may be provided to surround the tube section 402 along at least a portion of its length. The electrically conductive sleeve 420 may be made from a thin section of electrically conductive material, such as, for example, stainless steel. The conductive sleeve 420 can be attached in various ways including but not limited to, for example, via overmolding, ultrasonic welding, bonding with adhesives, interference fits or interference features between the sleeve 420 and the tube section 402 and/or other attachment mechanisms suitable for attaching a thin sleeve to the outer surface of the tube section 402. Alternatively, the conductive sleeve 420 could be captured by assembly order between the tube section 402 and either or both of the bowl 404 and the distal portion 410. In the exemplary embodiment shown, the sleeve 420 is provided at the proximal main portion 408. The sleeve 420 may be provided having a thickness so as to not significantly impact the structure (e.g., relative flexibility/rigidity) of the tube section 402. For example, in various exemplary embodiments, the thickness of the sleeve 420 may range from about 0.001 inch to about 0.010 inch. The sleeve 420 is disposed in a location and amount sufficient to allow electrical capacitive coupling of the cannula with a surgical instrument and the dissipation of the electrical capacitive charge, for example, so as to avoid alternate site burning, as will be discussed below in more detail with reference to FIGS. 5 and 6. In accordance with at least one exemplary embodiment, the bowl 404 is made from a polymer material.

While the exemplary embodiments discuss that the tube section and bowl are integrally connected with each other, it is to be understood that the tube section and bowl could be configured so as to be removable from each other.

FIG. 5 is a diagrammatic view of a portion of an exemplary robotic surgical system (it is noted that for simplicity the surgeon console and image processing/core computation unit are omitted, although those of ordinary skill in the art are familiar with such components of robotic surgical systems), with a cannula and surgical instrument shown in isolation, wherein the cannula is configured to achieve an electrical capacitive coupling with the surgical instrument and dissipate an electric charge in accordance with at least one exemplary embodiment of the present disclosure. The system includes a cannula 570 and one or more dispersive electrodes 560 (two being illustrated in FIG. 5). The system may also include an electrosurgical generator unit 510. The dispersive electrodes 560 are electrically coupled to the electrosurgical generator unit 510 through cables 520, for example. The cannula 570, in accordance with at least one embodiment, is disposable. The cannula 570 illustrated in FIG. 5 can be, for example, any one of cannulas 100, 200, 300, or 400, illustrated in FIGS. 1A-1B, 2A-2B, 3A-3B, and 4A-4B.

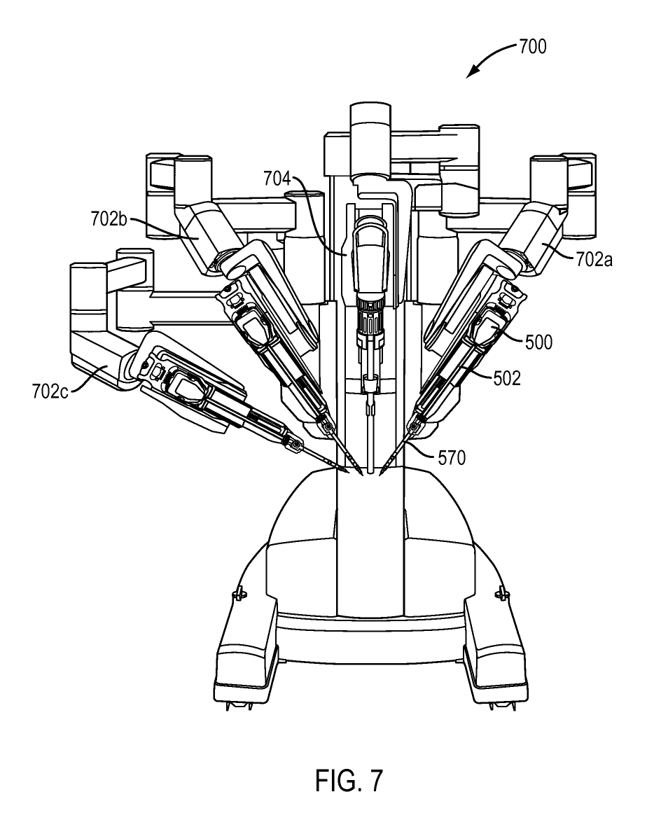

An electrosurgical instrument 500 is provided and, in operation, is inserted into the interior passage (e.g., such as interior passages 106, 206, 306, and 406) of the cannula 570 by, for example, instrument manipulators 702a-702c of a patient side cart 700 (see FIG. 7). The cannula 570 may be sufficiently flexible to permit some curving of the cannula 570 as the electrosurgical instrument 500 is inserted into the cannula 570. The patient side cart 700 is positioned proximate to a patient, and the surgical instrument 500, which includes a shaft 502, and in the embodiment depicted also may include a wrist 504, is used to perform various surgical procedures at a work site in the patient's body through the use of a remotely actuated end effector 506. Exemplary surgical procedures that the end effector 506 can perform include, but are not limited to, stapling, cutting, delivery of electrical energy (e.g., to cauterize and/or ablate), suturing, clamping, and combinations thereof.

In use with the system including the cannula 570, the dispersive electrodes 560, and the generator unit 510, a patient 550 is positioned on an operating table 552, and the cannula 570 is attached to the body of the patient 550 at an entry site 554. The dispersive electrodes 560 are disposed beneath the patient 550, for example, beneath the patient's shoulders and buttocks, or at other locations that provide sufficient surface area contact between the electrodes 560 and the patient's body so as to permit electrical conductance therebetween. The dispersive electrodes 560 therefore contact the body of the patient 550 when the patient 550 is positioned on the operating table 552.

The electrosurgical instrument 500 is inserted into the interior passage of the cannula 570 and is in electrical communication with the electrosurgical generator unit 510. The electrosurgical generator unit 510 supplies energy 530 to the instrument 500. An electrical charge passes through the instrument shaft 502 to the end effector 506, for example, via an insulated electrically conductive cable. The electrically conductive material of the cannula 570 is arranged and disposed in an amount sufficient to receive a charge 540 that is built up on the surgical instrument 500 via electrical capacitive coupling of the surgical instrument 500 and the cannula 570. For example, an amount of the material that is sufficient to receive a charge via electrical capacitive coupling may be sufficient so as to receive a current ranging from about several hundred milliamps (mA) or less, for example, about 50 milliamps (mA) or less, or for example, of at least about 10 milliamps (mA). The higher end of the range is generally associated with manual surgical laparoscopic instruments which may not include exposed metal parts other than the live electrical element, and the lower end of the range is generally associated with robotic surgical instruments. In the exemplary embodiment wherein the cannula 570 is configured as cannula 100, illustrated in FIGS. 1A-1B, the electrical charge that may build up on the surgical instrument, for example, on a wrist or other portion, is received by the stainless steel tube section 108. In the exemplary embodiment wherein the cannula 570 is configured as cannula 200, illustrated in FIGS. 2A-2B, the electrical charge is received by the one or more layers 220. In the exemplary embodiment wherein the cannula 570 is configured as cannula 300, illustrated in FIGS. 3A-3B, the electrical charge is received by the electrically conductive composite material 320. Finally, in the exemplary embodiment wherein the cannula 570 is configured as cannula 400, illustrated in FIGS. 4A-4B, the electrical charge is received by the sleeve 420 disposed about the tube section 408.

Regardless of the specific embodiment of the cannula 570, in accordance with the present disclosure, the electrical charge that is built up on the surgical instrument will be received by the cannula 570 through capacitive coupling in light of the incorporation of the electrically conductive material in the cannula 570's structure. Further, in light of the electrically conductive material incorporated in the cannula 570, the electrical charge received can be dissipated along the cannula 570 and through the patient's body due to the dispersive electrodes 560. The dissipation can be sufficient to inhibit and/or prevent the electrical charge from arcing from the surgical instrument to undesirable locations, for example, on the patient (potentially causing alternate site burning) and/or to other individuals in the surgical area. Current 545 passes from the dispersive electrodes 560 to the generator unit 510 through the cables 520. Further, if an electrical charge is built up at the wrist 504, the charge is transferred to the electrically conductive portion of the cannula 570 (or portion thereof), and is dissipated by the patient's body in contact with the dispersive electrodes 560.

FIG. 6 is a diagrammatic view of an exemplary robotic surgical system, with a cannula and instrument shown in isolation. The system utilizes the cannula to dissipate an electric charge in accordance with at least one exemplary embodiment of the present disclosure. Elements of FIG. 6 are similar to elements of FIG. 5. FIG. 6 shows the patient side cart 700 positioned to introduce instruments through the patient's mouth and into the oral cavity (the patient's neck is typically extended). In the embodiment of FIG. 6, the cannula 570 is provided in a position above the surgical site and does not contact the patient 550. The cannula 570 illustrated in FIG. 6 can be any one of cannulas 100, 200, 300 or 400, illustrated in FIGS. 1A-1B, 2A-2B, 3A-3B, and 4A-4B. The dispersive electrodes 560a, 560b are disposed beneath the patient 550, for example, beneath the patient's shoulders and buttocks, or at other locations that provide sufficient surface area contact between the electrodes 560a, 560b and the patient's body so as to permit electrical conductance therebetween. The dispersive electrodes 560a, 560b therefore contact the body of the patient 550 when the patient 550 is positioned on the operating table 552. The dispersive electrode 560a is also electrically coupled to an electrosurgical generator unit 510 through a cable 520. The dispersive electrode 560b is electrically coupled to the cannula 570 through an electrical connector 603 at the cable 600 that connects with an electrical connector 603 of the cannula 570. This connection forms an electrical contact between the patient 550 and the cannula 570. An electrically conductive path is thus created from the cannula 570 to the patient 550 through the dispersive electrode 560b, and to the electrosurgical generator unit 510 from the dispersive electrode 560a.

The instrument 500 is electrically coupled to the electrosurgical generator unit 510, which supplies energy to the instrument 500. An electrical charge passes through the instrument shaft 502 to the end effector 506, for example, via an insulated electrically conductive cable. The electrically conductive material of the cannula 570 is arranged and disposed in an amount sufficient to receive a charge 540 that is built up on the surgical instrument 500 via electrical capacitive coupling of the surgical instrument 500 and the cannula 570. In the exemplary embodiment wherein the cannula 570 is configured as cannula 100, illustrated in FIGS. 1A-1B, the electrical charge that may build up on the surgical instrument, for example, on a wrist or other portion, is received by the stainless steel tube section 108. In the exemplary embodiment wherein the cannula 570 is configured as cannula 200, illustrated in FIGS. 2A-2B, the electrical charge is received by the one or more layers 220. In the exemplary embodiment wherein the cannula 570 is configured as cannula 300, illustrated in FIGS. 3A-3B, the electrical charge is received by the electrically conductive composite material 320. Finally, in the exemplary embodiment wherein the cannula 570 is configured as cannula 400, illustrated in FIGS. 4A-4B, the electrical charge is received by the sleeve 420 disposed about the tube section 408. The electrical charge 604 is dissipated from the conductive material of the cannula 570 through the cable 600 to the dispersive electrode 560b to the patient's body 550. Further, if an electrical charge is built up at the wrist 504, the charge is transferred to the conductive portion of the cannula 570, which is dissipated by the patient's body 550 in contact with the dispersive electrodes 560b. In an alternative implementation, the cannula 570 may be electrically coupled directly to the generator unit 510, as depicted by the dashed line, or to a separate reference potential (not shown). From the patient's body, electrical charge 545 may be dissipated from the dispersive electrode 560a through the cable 520 to the electrosurgical generator unit 510.

In various exemplary embodiments, cannulas in accordance with the present disclosure may be configured as a flared cannula, for example, in an electrical charge-dissipating system as shown and described, for example, in U.S. patent application Ser. No. 12/946,693 (filed Nov. 15, 2010; disclosing “CANNULA”; claiming the benefit of U.S. Patent Application No. 61/387,843 (filed Sep. 29, 2010)), which is incorporated by reference in its entirety herein.

FIG. 7 is a diagrammatic view of a patient side cart 700 of an exemplary robotic surgical system in accordance with an exemplary embodiment of the present disclosure and with which the cannulas according to various exemplary embodiments described herein can be used. For simplicity in illustration, the associated surgeon console and the image processing/core computation unit are not shown; however, those of ordinary skill in the art are generally familiar with those additional components of robotic surgical systems. The depicted embodiment is a da Vinci® robotic surgical system patient side cart. The patient-side cart 700 includes three telerobotic instrument manipulators 702a-702c and a single telerobotic endoscopic camera manipulator 704 to which an endoscope and camera are attached. A cannula 570 is shown mounted at the end of the instrument manipulator 702a to illustrate how the cannula 570 is positioned with reference to other system components. One or more similar cannulas 570 may be mounted on manipulators 702b and/or 702c. A removable teleoperated minimally invasive surgical instrument 500 is illustratively shown mounted on the instrument manipulator 702a so that the instrument shaft 502 extends through the cannula 100. The surgical instrument 500's end effector 506 extends beyond the cannula 100's distal end. During surgery, one or more instrument manipulators are positioned to place the surgical instrument end effectors at a surgical work site. The surgeon controls the positions and orientations of the various surgical end effectors and the camera by making teleoperation master control inputs at the surgeon side console.

As described above, the apparatus, system and method in accordance with various exemplary embodiments can be used in conjunction with a surgical instrument having an end effector configured to perform multiple surgical procedures.

Other embodiments of the invention will be apparent to those skilled in the art from consideration of the specification and practice of the present disclosure and claims herein. It is intended that the specification and examples be considered as exemplary only, with a true scope and spirit of the invention being indicated by the following claims.

What is claimed is:

1. A cannula for receiving a surgical instrument to perform a surgical operation on a body, the cannula comprising:
a hollow elongated structure having a proximal end opening and a distal end opening leading to a hollow interior passage dimensioned to receive a surgical instrument, wherein the hollow elongated structure comprises:
a polymer material, and
an electrically conductive material, the electrically conductive material being disposed to achieve electrical capacitive coupling with the surgical instrument and to dissipate an electrical charge received via the electrical capacitive coupling.

2. The cannula of claim 1, wherein the hollow elongated structure further comprises:
a tube section configured to receive the surgical instrument, and
a bowl disposed at a proximal end of the tube section, the bowl sealing the surgical instrument to the cannula when the surgical instrument is received within the hollow elongated shaft.

3. The cannula of claim 2, wherein the bowl comprises the proximal end opening.

4. The cannula of claim 2, wherein at least the bowl comprises the plastic material.

5. The cannula of claim 4, wherein the tube section comprises the polymer material and the electrically conductive material.

6. The cannula of claim 4, wherein the tube section comprises only the electrically conductive material.

7. The cannula of claim 1, wherein the hollow elongated structure comprises a composite material comprising the polymer material and the electrically conductive material.

8. The cannula of claim 7, wherein the composite material comprises a matrix of the polymer material with the electrically conductive material dispersed in the matrix.

9. The cannula of claim 7, wherein the electrically conductive material comprises a fibrous electrically conductive material.

10. The cannula of claim 7, wherein the electrically conductive material comprises electrically conductive particulate material.

11. The cannula of claim 7, wherein the electrically conductive material is at least one of carbon and graphite.

12. The cannula of claim 2, wherein the electrically conductive material is provided at the tube section.

13. The cannula of claim 2, wherein the tube section comprises the polymer material and the electrically conductive material.

14. The cannula of claim 13, wherein the electrically conductive material is provided as at least one layer on an exterior surface of the tube section.

15. The cannula of claim 13, wherein the electrically conductive material is provided as at least one layer on an interior surface of the tube section.

16. The cannula of claim 2, wherein the tube section comprises the electrically conductive material.

17. The cannula of claim 1, wherein the electrically conductive material is chosen from graphite, stainless steel, and carbon.

18. The cannula of claim 1, wherein the electrically conductive material is of an amount sufficient to dissipate the electrical charge from the surgical instrument at a rate sufficient to inhibit arcing of the electrical charge from the instrument to a location proximate the instrument and outside of the cannula.

19. A system to dissipate an electrical charge from a surgical instrument, comprising:
a cannula, comprising:
a hollow elongated structure configured to receive a surgical instrument, at least a portion of the hollow elongated structure comprising a polymer material, and
an electrically conductive material provided at the hollow elongated structure sufficient to receive an electrical charge from the surgical instrument through capacitive coupling between the surgical instrument and the hollow elongated structure; and
at least one dispersive electrode configured to be placed in contact with a patient's body.

20. The system of claim 19, wherein the electrically conductive material is disposed to dissipate an electrical charge from the surgical instrument at a rate sufficient to inhibit arcing of the electrical charge from the instrument to a location proximate the instrument and outside of the cannula.

21. The system of claim 19, wherein the at least one dispersive electrode is configured to be electrically coupled to the cannula.

22. The system of claim 19, further comprising an energy generator unit electrically coupled to the surgical instrument to provide energy to the surgical instrument and electrically coupled to the at least one dispersive electrode.

23. The system of claim 19, wherein the hollow elongated structure comprises:
a tube section configured to receive the surgical instrument, and
a bowl disposed at a proximal end of the tube section, the bowl sealing the surgical instrument to the cannula when the surgical instrument is received within the hollow elongated structure.

24. The system of claim 23, wherein the electrically conductive material is provided as at least one layer on a surface of the tube section.

25. The system of claim 23, wherein the tube section comprises the electrically conductive material.

26. The system of claim 19, wherein the hollow elongated structure comprises a composite material comprising the polymer material and the electrically conductive material, the composite material comprising a matrix of the polymer material with the electrically conductive material dispersed in the matrix.

27. A method of dissipating an electrical charge from a surgical instrument, comprising:
connecting a cannula to a body in contact with at least one dispersive electrode, the cannula having an electrically conductive material provided at the cannula sufficient to receive an electrical charge from the surgical instrument through capacitive coupling between the surgical instrument and the cannula, at least a portion of the cannula comprising a polymer material;
inserting the surgical instrument within an interior passage of the cannula; and
providing energy to the surgical instrument from an energy source,
wherein the electrical charge is dissipated through the electrically conductive material of the cannula to the body after the surgical instrument is provided with energy from the energy source.

28. The method of claim 27, wherein the connecting the cannula to the body comprises directly attaching the cannula to the body, wherein the cannula is electrically connected to the at least one dispersive electrode directly through the body when the at least one dispersive electrode is in contact with the body.

29. The method of claim 27, wherein the connecting the cannula to the body comprises positioning the cannula at a distance from the body and connecting the cannula to the at least one dispersive electrode through an electrically conductive cable.

30. The method of claim 27, wherein the electrically conductive material dissipates the electrical charge from the surgical instrument at a rate sufficient to inhibit arcing of the electrical charge from the instrument to a location proximate the instrument and outside of the cannula.
